(19) United States
(12) Patent Application Publication
(10) Pub. No.: US 2015/0366501 A1
(43) Pub. Date: Dec. 24, 2015

(54) WHITE MATTER DIFFUSION TENSOR IMAGING TEST TO PREDICT TREATMENT OUTCOMES IN MEDICAL TREATMENT

(71) Applicant: BRC OPERATIONS PTY LIMITED, Ultimo, New South Wales (AU)

(72) Inventors: Evian Gordon, Vaucluse (AU); Stuart Grieve, Ultimo (AU); Mayuresh Korgaonkar, Ultimo (AU)

(73) Assignee: BRC Operations Pty Limited, Ultimo (AU)

(21) Appl. No.: 14/763,875

(22) PCT Filed: Jan. 28, 2014
(86) PCT No.: PCT/AU2014/000045

§ 371 Date: Jul. 28, 2015

Related U.S. Application Data
(60) Provisional application No. 61/757,568, filed on Jan. 28, 2013.

(51) Int. Cl. A61B 5/00; G01R 33/48; A61K 31/343; A61K 31/137; A61B 5/055; A61B 5/16

(52) U.S. Cl. CPC A61B 5/4848; A61B 5/4064; A61B 5/055; A61B 5/165; A61K 31/343; A61K 31/137; G01R 33/4806

(57) ABSTRACT

The present invention relates to the field of biomarkers and in particular to their utilisation in treatment. Embodiments of the invention have been particularly developed as biomarkers enabling optimisation of treatment regimes and as uses of the biomarkers in tests for the prediction of optimised treatments and treatment outcomes in the treatment of Major Depressive Disorder (MDD).

### CROSS-REFERENCE

This application incorporates herein by cross-reference U.S. provisional patent application 61/757,568 filed 28 Jan. 2013 in its entirety.

### FIELD OF THE INVENTION

The present invention relates to the field of biomarkers and in particular to their utilisation in treatment. Embodiments of the invention have been particularly developed as biomarkers enabling optimisation of treatment regimes and as uses of the biomarkers in tests for the prediction of optimised treatments and treatment outcomes in the treatment of Major Depressive Disorder (MDD). The invention will be described hereinafter with reference to this application. However, it will be appreciated that the invention is not limited to this particular field of use.

### BACKGROUND

Any discussion of the background art throughout the specification should in no way be considered as an admission that such art is widely known or forms part of common general knowledge in the field.

Major depressive disorder (MDD) affects 121 million people worldwide. It has the highest burden of illness in high-income countries based on disability-adjusted life-years, is the third most disabling medical condition worldwide and is the second-ranked cause of lost quality of life in persons aged 15 to 44 years and is expected to move to second by 2020 (World Health Organization. Mental Health: Mental Health Atlas 2011. Switzerland: World Health Organization, 2011). The economic burden of depression in the United States in 2000 amounted to roughly 83 billion US dollars, 31% of which were attributed to direct medical costs (Greenberg at al. 2003).

Antidepressant medications (ADMs) are the most commonly-used treatment for depression, especially in primary care. ADMs are effective (American Psychiatric Association. Practice guideline for major depressive disorder in adults. Am J Psychiatry 1993; 150(4 Suppl.):1-26; Depression Guideline Panel. Clinical practice guideline. Number 5. Depression in primary care: volume 2. Treatment of major depression. Rockville, Md.: U.S. Department of Health and Human Services, Public Health Service, Agency for Health Care Policy and Research, 1993; Frank E. et al., 1993; These, M. E. and Rush, A. J. Treatment-resistant depression. In: Bloom F E, Kupfer D J, eds. Psychopharmacology: fourth generation of progress. New York: Raven Press, 1995:1081-97; Rosenbaum J. F. et al., Treatment-resistant mood disorders. In: Gabbard G O, ed. Treatments of psychiatric disorders. 3rd ed. Washington, D.C.: American Psychiatric Press, Inc., 2001:1307-84), but their benefit could be improved in at least two ways.

The first would be to identify pre-treatment clinical or neurobiological features that predict response versus non-response to any single treatment (predictors). Currently, ≦50% of patients reach remission following any single first-line antidepressant (Frank E. et al., Conceptualization and rationale for consensus definitions of terms in major depressive disorder. Remission, recovery, relapse and recurrence. Arch Gen Psychiatry 1991;48:851-5; Fava M. and Davidson K. G. Definition and epidemiology of treatment-resistant depression. Psychiatr Clin North Am 1996;19:179-200; Bileski R. J at al., A double-blind comparison of escitalopram and venlafaxine extended release in the treatment of major depressive disorder. J Clin Psychiatry 2004;65:1190-6; Rush A. J. et al., Acute and longer-term outcomes in depressed outpatients requiring one or several treatment steps: a STAR*D report. Am J Psychiatry 2006;163:1905-17; Trivedi M. H. et al., Evaluation of outcomes with citalopram for depression using measurement-based care in STAR*D: implications for clinical practice. Am J Psychiatry 2006;163:28-40; Gartlehner G. et al., Comparative benefits and harms of second-generation antidepressants: Background paper for the American College of Physicians. Ann Intern Med 2008;149:734-50), and gains in response and remission at second and subsequent steps are even more limited.

The second would be to identify features that recommend one treatment over another (so-called moderators) to find the best match between patient and treatment.

In addition to depressed mood, the diagnostic symptom criteria for Major Depressive Disorder (MDD) include impaired cognitive behaviours. These behaviours encompass memory and concentration, and impaired emotional behaviours such as a bias to negative emotion (magnification of threat). Cognitive and emotional behaviours are subserved by the frontal, limbic and brainstem circuits which are also involved in the action of antidepressants, including serotonin and norepinephrine and interactions with dopamine (e.g. Austin et al., 2001).

Currently, there is no objective test for predicting response outcomes based on the patient's pre-treatment profile. Attempts have been made to identify clinical- or laboratory-based patient- or disease-specific characteristics to assist in treatment selection (Morishita S. and Kinoshita T. Predictors of response to sertraline in patients with major depression. Human Psychopharmacology 2008;23:647-51; Papakostas G. I. and Fava M. Predictors, moderators and mediators (correlates) of treatment outcome in major depressive disorder. Dialogues in Clinical Neuroscience 2008;10:439-51; Kuk A. Y at al., Recursive subsetting to identify patients in the STAR*D: A method to enhance the accuracy of early prediction of treatment outcome and to inform personalized care. J Clin Psychiatry 2010; 71:1502-8) but current evidence is insufficient to be incorporated into guideline recommendations (Rush A. J. et al, Selecting among second-step antidepressants medication monotherapies: predictive values of clinical, demographic, or first-step treatment features. Arch Can Psychiatry 2008;8:870-80).

Furthermore, previous studies fail to disclose results showing which particular ADM is to be selected with confidence that administration of the selected ADM to an MDD patient will, in fact, lead to beneficial treatment outcome. As such, due to this lack of reliable predictors of treatment outcomes, ADMs are currently still being prescribed on the basis of trial and error.

To illustrate the consequences, it is noted that in general practice a period of about 6 to 8 weeks is required to assess the effectiveness of the ADM (if any) for each individual patient and that, should the first-chosen ADM prove to have been ineffective, a further ADM will be prescribed/tried. The possibility of having to try a several ADMs over a period spanning many months before any treatment effect is observed places a great burden and stress on a patient seeking relief from the symptoms of MDD. As such, an urgent need exists to identify biomarkers which can serve as valid and reliable predictors of treatment outcomes in patients suffering from symptoms of MDD.

While predictors of remission or moderators of treatment response are highly sought after, it is noteworthy that the accurate pre-treatment identification of a meaningful proportion of depressed outpatients who will not remit when being treated with ADMs is also very important when attempting to reduce time, effort, cost and patient burden associated with the treatment of MDD.

The prediction of non-remission must be accurate enough for the clinician to take action—i.e. accurate enough to allow the clinician to decide against the administration of an ADM (Kuk A Y, Li J, Rush A J: Recursive subsetting to identify patients in the STAR*D: a method to enhance the accuracy of early prediction of treatment outcome and to inform personalized care. J Clin Psychiatry 2010, 71(11):1502-1508),

In other words, the specificity of the prediction of non-remission must be high (e.g. over 85-90%) in order to ensure that only few are denied a treatment that has a reasonable chance of success. A reliable predictor of non-remission could move patients to later steps in the treatment program (e.g. somatic or psychotherapeutic treatments) without having to undergo ADM administration without significant therapeutic benefit (Li J, Kuk A Y, Rush A J: A practical approach to the early identification of antidepressant medication non-responders. Psychol Med 2011:1-8).

### SUMMARY OF THE INVENTION

It is an object of the present invention to provide a test for the prediction of treatment outcomes in MDD based on DTI imaging.

Pre-treatment functional activity in the anterior cingulate cortex has shown promise as a predictive marker of response to single treatments in MDD (Pizzagalli Da: Frontocingulate dysfunction in depression: toward biomarkers of treatment response. Neuropsychopharmacology: official publication of the American College of Neuropsychopharmacology 2011, 36:183-206; Siegle G J, Thompson W K, Collier A, Berman S R, Feldmiller J, Thase M E, Friedman E S: Toward clinically useful neuroimaging in depression treatment: prognostic utility of subgenual cingulate activity for determining depression outcome in cognitive therapy across studies, scanners, and patient characteristics. Archives of general psychiatry 2012, 69(9):913-924).

One technique to investigate the structural white matter connectivity in neural circuits is DTI. Examples of using DTI in the context of MDD can be found in Korgaonkar M S, Cooper N J, Williams L M, Grieve S M: Mapping inter-regional connectivity of the entire cortex to characterize major depressive disorder: a whole-brain diffusion tensor imaging tractography study. Neuroreport 2012:566-571; and Korgaonkar M S, Grieve S M, Koslow S N, Gabrieli J D E, Gordon E, Williams L M: Loss of white matter integrity in major depressive disorder: evidence using tract-based spatial statistical analysis of diffusion tensor imaging. Human brain mapping 2011, 32:2161-2111.

The present inventors are involved in an international clinical study in which a large group of outpatients with non-psychotic MDD has been examined with a view to identifying predictors for optimised treatment in depression. The study has been designed as a real-world effectiveness trial, primarily to identify which pre-treatment characteristics could serve as the long-sought predictors or moderators of treatment response to commonly-used antidepressants or antidepressant types.

The goal of the international clinical study was to identify predictors or moderators of treatment outcomes that are sufficiently predictive to change how practitioners select among ADMs when treating MDD. The present inventors have found surprising, robust correlations between certain pre-treatment patient characteristics and desired treatment outcomes with respect to the three medications most commonly prescribed as first-line ADMs world-wide, namely escitalopram, sertraline and venlafaxine-extended release (venlafaxine-XR). Escitalopram and sertraline are both selective serotonin reuptake inhibitors (SSRIs) wherein venlafaxine-XR is a selective norepinephrine and serotonin reuptake inhibitor (SNRI).

Diffusion tensor imaging (DTI) is a non-invasive MRI technique that measures the connectivity within white matter tracts in vivo by calculating the direction and magnitude of water diffusion within the brain (fractional anisotropy; FA). DTI allows for the characterisation of the integrity of WM tracts of the brain and can measure abnormalities in brain circuitry present in MDD patients (Korgaonkar M S, Cooper N J, Williams L M, Grieve S M: Mapping inter-regional connectivity of the entire cortex to characterize major depressive disorder: a whole-brain diffusion tensor imaging tractography study, Neuroreport 2012:566-571; Korgaonkar M S, Grieve S M, Koslow S H, Gabrieli J D E, Gordon E, Williams L M: Loss of white matter integrity in major depressive disorder: evidence using tract-based spatial statistical analysis of diffusion tensor imaging. Human brain mapping 2011, 32:2161-2171). In some cases these abnormalities can be correlated with treatment response and thus identify individual patients whose brain networks are either favourable or unfavourable for the mode of action of a particular ADM, As such, DTI has been found to identify disruptions to white matter connectivity that predict ADM treatment outcomes.

The frontal-limbic circuits, specifically the amygdala-hippocampal complex and the anterior cingulate region, are central to our understanding of clinical depression and the action of antidepressant medications (Canli T, Cooney R E, Goldin P, Shah M, Sivers H, Thomason M E, Whitfield-Gabrieli S, Gabrieli J D E, Gotlib I H: Amygdala reactivity to emotional faces predicts improvement in major depression. Neuroreport 2005, 16:1267-1270; Mayberg H S, Brannan S K, Mahurin R K, Jerabek Pa, Brickman J S, Tekell J L, Silva Ja, McGinnis S, Glass T G, Martin C C et al: Cingulate function in depression: a potential predictor of treatment response. Neuroreport 1997, 8:1057-1061; Whalen P J, Johnstone T, Somerville L H, Nitschke J B, Polis S, Alexander A L, Davidson R J, Kalin N H: A functional magnetic resonance imaging predictor of treatment response to venlafaxine-XR in generalized anxiety disorder. Biol Psychiatry 2008, 63(9):858-863).

In the development of a DTI-based test for the prediction of treatment outcome in MDD patients, the inventors evaluated the utility of the DTI measures of WM integrity of anterior cingulate and limbic circuits in predicting response to three commonly prescribed antidepressant drugs—escitalopram, sertraline and venlafaxine-XR.

In a first aspect, the present invention relates to a method of determining a medical treatment outcome in a brain-related condition of a subject, the method including the steps of:
a) determining the state of the white matter circuits of the subject's brain; and
b) utilising said state of the white matter circuits to predict the likely medical treatment outcome.

The determining step can include conducting DTI of the subject's brain. The step of utilising can include determining the state of anterior cingulate or the limbic circuits as a biomarker for the prediction of the likely medical treatment outcome. In some embodiments the degree of connectivity of the cingulate portion of the cingulum and the stria terminalis are utilised in the determining step.

Typically, the brain-related condition is Major Depressive Disorder (MDD).

In some embodiments, the treatment is a treatment for depression. Typically the treatment for depression comprises the administration of an ADM. The medical treatment can include the administration escitalopram, sertraline and venlafaxine-extended release (venlafaxine-XR). The medical treatment is preferably the administration of venlafaxine-XR.

In some embodiments the invention relates to a DTI test of the integrity of the white matter tracts of specific brain regions for the prediction of a reduction of symptom presentation in patients diagnosed with MDD and treated with venlafaxine-XR.

Typically, DTI imaging of the fractional anisotropy (FA) of the cingulate gyros and the stria terminalis is performed. In 102 depressed outpatients, DTI imaging of the FA of the cingulate gyros and the stria terminalis, predicted remission with an acceptable degree of specificity (80%) and sensitivity (57%). As such, in some embodiments, combination of higher connectivity (greater FA values) for the cingulate portion of the cingulum and lower FA values for the stria terminals are predictive of symptom remission and a combination of higher FA values for the cingulate portion of the cingulum and lower FA values for the fornix are predictive of symptom response.

Conversely, in other embodiments, non-remission was found to be associated with lower connectivity (lower FA) in the cingulate region and greater connectivity (greater FA) in the stria terminalis (which contains amygdala outflow).

Treatment outcome is routinely determined by a symptom score according to the clinician-rated 17-item Hamilton Rating Scale for Depression (HRSD₁₇) or the self-rated the 16-item Quick Inventory of Depressive Symptomatology-Self Report (QIDS-SR₁₆), wherein a ≧50% decrease of a symptom score determined before treatment with a selected ADM after 8 weeks of treatment with said selected ADM indicate a treatment response and wherein symptom scores of ≦7 on the HRSD₁₇ or of ≦5 on the QIDS-SR₁₆ after 8 weeks of treatment with said selected ADM indicate remission.

Typically, the ADM is selected from the group consisting of selective serotonin reuptake inhibitors (SSRIs) and serotonin reuptake inhibitors (SNRIs).

In some embodiments the ADM is selected from escitalopram, sertraline and venlafaxine-extended release (venlafaxine-XR).

In a second aspect, the present invention relates to a method of predicting symptom remission or symptom response in a patient with Major Depressive Disorder (MDD) when treated with a selected ADM comprising the steps of:
a) performing diffusion tensor imaging (DTI) of the cingulate gyrus and the stria terminalis to measure fractional anisotropy (FA) of the white matter circuits in these areas of the subject's brain; and
b) utilising the measure of step a) to predict symptom remission or symptom response,
   wherein a ratio of FA values for the cingulate portion of the cingulum and FA values for the stria terminals below 1 is predictive of symptom remission.

In a third aspect, the present invention relates to a method of predicting non-remission in a patient with Major Depressive Disorder (MDD) when treated with a selected ADM comprising the steps of:
c) performing Diffusion Tensor Imaging (DTI) of the cingulate gyrus and the stria terminalis to obtain a measure of fractional anisotropy (FA) in these areas of the subject's brain; and
d) utilising said measure of step a) to predict non-remission,
   wherein a ratio of FA values for the stria terminalis and FA values for the cingulate portion of the cingulum below 1 is predictive of non-remission.

In a fourth aspect the present invention relates to a method of treating Major Depressive Disorder (MDD) in a patient, wherein said MDD is associated with a particular state of the white matter circuits of the subject's brain, said method comprising the steps of:
a) performing diffusion tensor imaging (DTI) to obtain a measure of fractional anisotropy (FA) in an area of the subjects brain;
b) comparing said measure of a) with a reference set of FA measures obtained from MDD patients to establish a correlation between said measure of a) with a corresponding FA measure of the reference set;
c) selecting an antidepressant medication (ADM) based on said correlation of step b), and wherein said corresponding FA measure of the reference set has been linked to beneficial treatment outcome in MDD patients having been treated with said ADM; and
d) administering said ADM selected in c) to said patient to treat said MDD.

Typically, DTI of the cingulate gyrus and the stria terminalis is performed in step a) above.

Typically, the ADM is selected from the group consisting of selective serotonin reuptake inhibitors (SSRIs) and serotonin reuptake inhibitors (SNRIs).

In some embodiments the ADM is selected from escitalopram, sertraline and venlathxine-extended release (venlafaxine-XR).

Also, for methods of the present invention, the regression model is a univariate regression model. In some embodiments, the univariate regression model includes running separate univariate models for each of the genomic parameters, incorporating cross-validation using a k-fold approach. In some alternative embodiments, the regression model is a multivariate logistic regression model. Generally, the regression model provides a statistical significance of p<0.01.

In the context of the present application the term “Major Depressive Disorder (MDD)” includes but is not limited to mood disorders such as clinical depression, major depression, unipolar depression, unipolar disorder or recurrent depression in the case of repeated episodes if diagnosed in accordance with the Diagnostic and Statistical Manual of Mental Disorders IV (DSM-IV published by the American Psychiatric Association (1994). Diagnostic and statistical manual of mental disorders (4^{th} ed). Washington D.C.). Depressive symptom severity of MDD can be rated using any appropriate rating scale including the 17-item Hamilton Rating Scale for Depression (HRSD₁₇) and the 16-item Quick Inventory of Depressive Symptomatology-Self Report (QIDS-SR₁₅).

In the context of the present application the term “biomarker” includes but is not limited to objectively measurable and assessable indicators of a biological process or biological state. Preferably, the biomarkers of the present invention indicate changes in symptom severity of MDD experienced by a patient in response to treatment. In accordance with the present invention, measurable and assessable cognitive and/or genomic parameters can be biomarkers indicating changes in symptom severity of MDD.

In the context of the present application the term “predictor of treatment outcome” includes but is not limited to biomarkers as defined above, which have a predictive quality with respect to the treatment outcome in MDD patients when treated with an antidepressant drug (ADM). “Biomarkers which have predictive quality with respect to the treatment outcome in MDD patients” here includes but is not limited to biomarkers which have been shown to be statistically significantly correlated with a change in treatment outcome in MDD patients.

In the context of the present application the term “treatment outcome” refers to certain threshold depression symptom scores measured by any appropriate rating scale, including the clinician-rated HRSD₁₇ and self-rated QIDS-SR₁₆ after treatment when compared to the symptom scores obtained before treatment. Typically, treatment outcome targets are: “symptom response”, defined for example as a ≧50% decrease from the baseline score for HRSD₁₇ or QIDS-SR₁₆ and “symptom remission”, defined for example as a score of ≦7 on the HRSD₁₇ or a score of ≦5 on the QIDS-SR₁₆.

In the context of the present application the term “statistically significant correlation” includes but is not limited to statistical correlations having p-values in a range of ≦0.05 (i.e. p-values of ≦0.01, ≦0.005, ≦0.001, ≦0.0005, or ≦0.0001), or accuracy/sensitivity/specificity in a range of 0.50 or greater (i.e. 0.55, 0.60, 0.65, 0.70, 0.75, 0.80, 0.85, 0.90, 0.95, 0.99 or greater), depending on the specific analysis and the most relevant values for evaluating the outcome of that analysis.

In the context of the present application the term “symptom score” includes but is not limited to any objective measure of symptom severity in MDD patients. Preferably, symptom scores are determined in accordance with the Diagnostic and Statistical Manual of Mental Disorders IV (DSM-IV published by the American Psychiatric Association (1994). Diagnostic and statistical manual of mental disorders (4^{th} ed). Washington D.C.) and measured by the clinician rated HRSD₁₇ and self-rated QIDS-SR₁₆.

In the context of the present application the term “antidepressant medication (ADM)” includes but is not limited to medications which regulate the balance of effectors of mood disorders such as neurotransmitters, for example serotonin, norephinephrine or dopamine. Preferably, the ADMs are selective serotonin reuptake inhibitors (SSRIs) or selective norephinephrine and serotonin reuptake inhibitors (SNRIs) such as escitalopram or sertraline and venlafaxine-XR, respectively.

In the context of the present application the term “a reference set of FA measures” includes but is not limited to a set of FA measures previously found to be statistically significantly correlated with a symptom score of MDD thereby providing a reference set of FA measures being statistically significant predictors of treatment outcome in MDD. The methods according to the present invention provide for the establishment of such a reference set. The reference set, which can also be an index, is useful in clinical practice to determine de nova treatment regimes with greater confidence of a beneficial treatment outcome for each individual patient as well as to determine optimized treatment regimes for patients with MDD already receiving ADM.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Embodiments of the invention will now be described, by way of example only, with reference to the accompanying drawings in which:

FIG. 1, in an upper panel, shows the white matter tracts and the white matter skeleton representing the centre of all white matter tracts overlaid on a standard brain (FIG. 1A and FIG. 1B). Lower panel shows baseline WM fractional anisotropy differences in the Cingulum Cingulate (CgC; FIG. 1C) and the Stria Terminalis (ST; FIG. 1D) between MDD patients who do and do not remit after 8 weeks of treatment with venlafaxine-XR (remitter and non-remitter patients, respectively).

FIG. 2 shows the distribution of R_{ST-Cing} for both the remitter and non-remitter patients. Both groups do not significantly deviate from a normal distribution (Shapiro-Wilk=0.98, p>0.5). The remitting population is skewed to the left (0.254), while the non-remitting population has a distribution slightly skewed to the right (−0.256). R_{ST-Cing} was significantly lower for remitters compared to non-remitters (0.93±0.05 versus 0.97±0.07, p<0.002). Using a threshold of R_{ST-Cing}>1.0 to select the non-remitter group, 31% of the overall non-remitters and 3% of overall remitters were selected. This corresponds to an NPV of 91%.

FIG. 3 is a flow diagram illustrating the sequence of steps required to perform a method of determining a medical treatment outcome in a brain-related condition of a subject in accordance with the present invention.

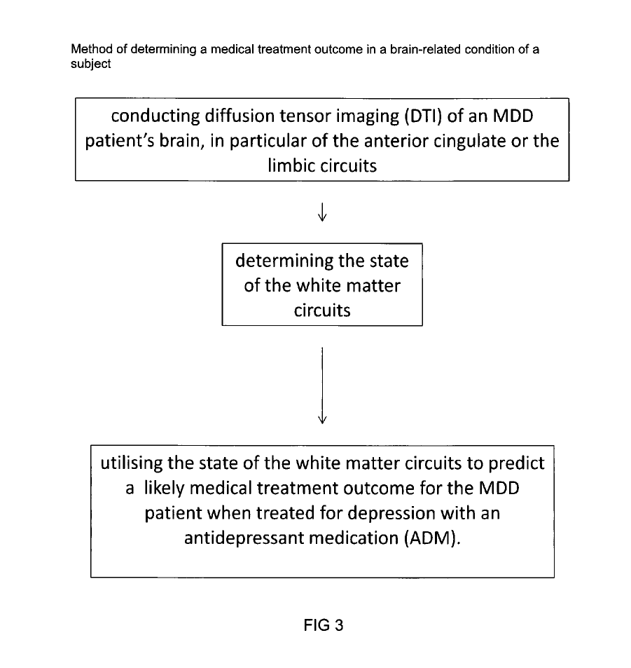

FIG. 4 FIG. 3 is a flow diagram illustrating the sequence of steps required to perform a method of predicting symptom remission or non-remission in a patient with Major Depressive Disorder (MDD) when treated with a selected ADM in accordance with the present invention

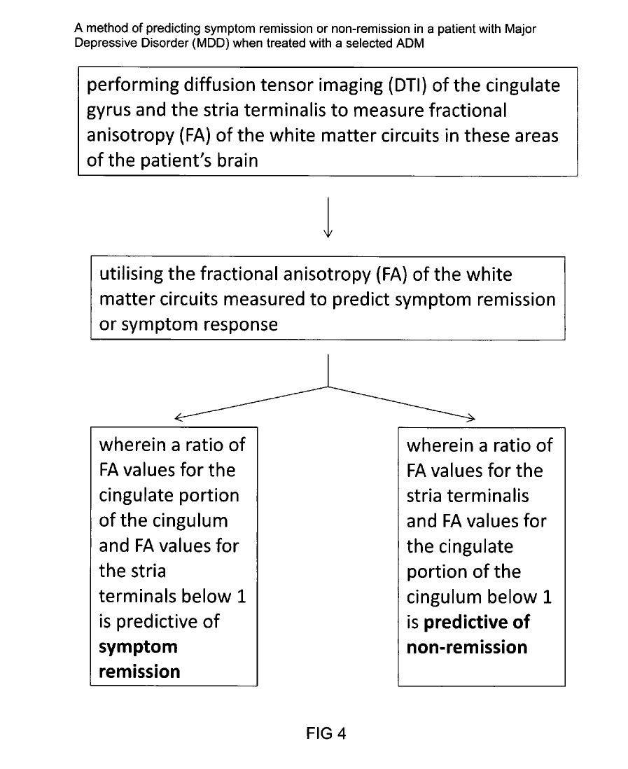

### DETAILED DESCRIPTION

Preferred embodiments of the invention will now be described.

In one preferred embodiment the present invention relates to a method of identifying a predictor of treatment outcome in Major Depressive Disorder (MDD).

At the outset, the Mini-International Neuropsychiatric Interview (MINI-Plus) (Sheehan D V, Lecrubier Y, Sheehan K H, Amorim P, Janays J, Weiller E, Hergueta T, Baker R, Dunbar G C: The Mini-International Neuropsychiatric Interview (M.I.N.I.): The development and validation of a structured diagnostic psychiatric interview for DSM-IV and ICD-10. J Clin Psychiatry 1998, 59(Suppl. 20):22-33) is used to confirm the criteria for non-psychotic MDD in accordance with the Diagnostic and Statistical Manual of Mental Disorders IV (DSM-IV published by the American Psychiatric Association 1994) and to assess for psychiatric and substance abuse disorders and other potential exclusion criteria. Depressive symptom severity can be rated using the 17-item Hamilton Rating Scale for Depression (HRSD-₁₇) and the 16-item Quick Inventory of Depressive Symptomatology-Self Report (QIDS-SR₁₆). HRSD₁₇ and QIDS-SR₁₆ scores are collected at baseline (i.e. before treatment) and after a predetermined period of treatment with a selected antidepressant medication (ADM), for example after 8 weeks. In some embodiments, the QIDS-SR₁₆ is collected at baseline and after 2, 4, 6 and 8 weeks. In other embodiments, the group of subjects with MDD is assigned into separate treatment groups for treatment with a selected antidepressant medication (ADM) and the HRSD₁₇ and QIDS-SR₁₆ scores are collected again collected as described above, The HRSD₁₇ and QIDS-SR₁₆ scores provide a measure of the degree of MDD in a subject with reference to the MDD symptom severity determined. The scores obtained in the above-mentioned symptom reporting scales are referred to herein as “symptom scores”.

The comparison of the symptom scores measured before and after treatment with the selected ADM provides a measure of treatment outcome for each patient. The skilled reader will, of course, understand that such symptom scores can be measured by any applicable diagnostic method for determining the degree of MDD known in the art and that the statistical comparison of the scores can also be performed by methods commonly known in the art.

Once the baseline symptom scores have been collected, and before any treatment with an ADM has commenced, further baseline parameters are established for each of the subjects with MDD as well as for subjects of a matched control group who do not suffer from MDD. Patients were recruited from an academic psychiatry setting, with recruitment from community and primary care physicians. The parameters assessed in one embodiment are, for example, the subjects' white matter circuitry of the cingulate portion of the cingulum (CgC) and hippocampus (CgH), the stria terminalis (ST), the fornix (FX) and the uncinate fasciculus (UNC) as analysed using DTI.

Diffusion Tensor Imaging (DTI) is a non-invasive MRI technique that allows characterizing of the integrity of white matter (WM) tracts of the brain such as the WM tracts of the anterior cingulate and limbic circuits.

The skilled addressee will understand that one technique to investigate the structural white matter connectivity in neural circuits is DTI. The measures of fractional anisotropy (FA) provide the basis of the diffusion tensor imaging of the white matter circuitry analysed and that, as such, the FA values provide a measure of the above-described parameters. The use of DTI to investigate the structural white matter connectivity in neural circuits in the context of MDD has been described in Korgaonkar et al. 2012 (Korgaonkar M S, Cooper N J, Williams L M, Grieve S M: Mapping inter-regional connectivity of the entire cortex to characterize major depressive disorder: a whole-brain diffusion tensor imaging tractography study. Neuroreport 2012:566-571) and Korgaonkar et al. 2011 (Korgaonkar M S, Grieve S M, Koslow S H, Gabrieli J D E, Gordon E, Williams L M: Loss of white matter integrity in major depressive disorder: evidence using tract-based spatial statistical analysis of diffusion tensor imaging. Human brain mapping 2011, 32:2161-2171).

In the present study, 102 MDD patients underwent imaging (DTI), with 34 matched patients in each of the three treatment arms. Notwithstanding, the skilled addressee will also appreciate that FA values are listed as examples only and that the present invention can be performed by assessing other measures of parameters relevant to MDD.

Once all baseline measures have been recorded, the group of subjects with MDD is assigned into separate treatment arms for treatment with different, selected ADMs. Following the predetermined period of treatment with the ADM, the above-described parameters are again assessed. As indicated above, the predetermined period of treatment in one embodiment of the present invention is 2, 4, 6 and 8 weeks. By assessing the parameters before treatment with one of the selected ADMs as well as during and/or after treatment with the selected ADM, FA values for each individual subject in each of the treatment groups are obtained.

In some embodiments of the present invention, for the purpose of analysing the symptom scores and FA values obtained, subjects with MDD are divided into at least two sub-groups based on a comparison of the FA values obtained for at least one of said parameters in the group of subjects with MDD before treatment with the FA values obtained for the same parameter in the control group. For example, a division into sub-groups based on FA values obtained for the subjects with MDD can be based on the FA values of the subjects with MDD being “high” or “low”; “average”, “above average” or “below average; “similar” or “dissimilar”; “better” or “poorer”; “slower” or “faster”; etc. in comparison to the FA values of the control group for the same parameter. The skilled addressee will appreciate that the above-mentioned division into sub-groups based on a statistical comparison of the FA values can be referred to as “splitting” “grouping”, “clustering”, “stratifying”, etc. the FA values/DTI data obtained and that this can be performed using routine and well-known statistical methods. In some preferred embodiments the division into sub-groups is selected from clustering, splitting into equal proportions, or from splitting on some attribute such as a chosen z-score level or a chosen raw score value.

The FA values obtained are analysed to establish a statistically significant correlation between a change in treatment outcome (indicated by a relative change in symptom scores measured before, during and/or after treatment) a change for at least one of the parameters mentioned above (indicated by a relative change in FA values obtained before, during and/or after treatment). In one embodiment of the present invention, when a change for a parameter is statistically significantly correlated with a change in treatment outcome across one of the treatment groups, this parameter has been identified as a predictor of treatment outcome in MDD.

In some preferred embodiments the statistical method to analyse the FA values to establish a statistically significant correlation between a relative change in symptom scores measured before, during and/or after treatment and a relative change in FA values obtained before, during and/or after treatment is selected from multivariate logistic regression, univariate logistic regression and linear discrimant analysis. However, the skilled reader will understand that the analysis of the FA values to establish the statistically significant correlation can be performed using other routine and well-known statistical methods.

Clinical study

All MDD patients were either ADM naive or had undergone a wash out period if previously prescribed an ADM. Patients were randomized to receive escitalopram, sertraline, or extended-release venlafaxine-XR. Investigators/“raters” and patients were not blind to treatment assignment. ADMs were prescribed and doses adjusted by the patient's treating clinician according to routine clinical practice, but following the recommended dose ranges. Patients returned for their follow-up visit at the end of 8 weeks. The HRSD₁₇ was used to assess depression severity at week 8. Twenty-three patients exited the study before the week 8 follow-up and did not have HRSD₁₇ scores at week 8. These scores were imputed using Schafer's method (Schafer J L: Analysis of incomplete multivariate data. London; Chapman & Hall; 1996.). The present inventors used the relationship between acquired values for the HRSD₁₇ at week 8 and the Quick Inventory of Depression Symptomatology (16-item, Self-Report) (CODS-SR16) taken at weeks 2, 4 and 6. To ensure a stable imputation, the variability in each of the imputed datasets was explicitly modelled and consistency between imputed and non-imputed data was demonstrated to indicate that this approach to missing data did not affect the result.

The primary outcome of the clinical study was remission to treatment determined by clinician-rated HRSD₁₇ as a scores ≦7. Response rate was also analysed and was defined as a a ≧50% decrease in severity from baseline to week 8. In addition, patient's age of onset and disease duration (years) was documented for analyses. This study was conducted according to the principles of the Declaration of Helsinki 2008. After the study procedures were fully explained in accordance with the ethical guidelines of the institutional review board, participants provided written informed consent.

Table 1 shows the clinical and demographic subject characteristics, together with the remission rates for participants with MDD. The average daily dose (mg/d) for the three treatment arms was: escitalopram=12±7; sertraline=53±28; and venlafaxine-XR=86±32. Remission rate across the whole group was 39.2%, and rates were similar across treatment arms (remission rate: 45.4% for 1008 MDD participants at the time of analysis). Remitters were younger and had shorter disease duration (Table 1).

TABLE 1
Demographics & clinical measures summary

| | | | Remission | |
|---|---|---|---|---|
| | Controls | MDD | Yes | No |
| N | 34 | 102 | 39.2% | 60.8% |
| | | | (40/102) | (62/102) |
| Age^{a} | 31.5 ± 12.4 | 33.8 ± 13.1 | 28.8 ± 7.7 | 37.1 ± 14.7 |
| No. of Females | 16 (47) | 48 (47.0) | 17 (42.5) | 31 (50.0) |
| (%) | | | | |
| HRSD₁₇Baseline^{a} | 1.0 ± 1.2 | 21.0 ± 3.9 | 21.5 ± 4.3 | 20.7 ± 3.6 |
| HRSD₁₇ Week 8 | 1.1 ± 1.5 | 9.3 ± 4.8 | 4.7 ± 1.8 | 12.4 ± 3.7 |
| HRSD₁₇ % | | 54.4 ± 24.9 | 77.4 ± 9.7 | 39.1 ± 19.6 |
| change | | | | |
| Age of Onset^{a} | | 22.1 ± 12.2 | 19.6 ± 7.7 | 23.7 ± 14.2 |
| Disease duration | | 11.3 ± 11.8 | 8.6 ± 7.5 | 13.0 ± 13.7 |

^{a}Difference between Remitters and Non-remitters at p < 0.05

The DTI data obtained are analysed to establish a statistically significant correlation between a change in treatment outcome (indicated by a relative change in symptom scores measured before, during and/or after treatment) a change for at least one of the parameters mentioned above (indicated by a relative change in FA measure obtained before, during and/or after treatment). In one embodiment of the present invention, when a change in the DTI data (i.e. FA values) for one of the analysed brain regions is statistically significantly correlated with a change in treatment outcome across one or all of the treatment groups, this change in white matter circuitry for the region has been identified as a predictor of treatment outcome in MDD.

In some preferred embodiments the statistical method to analyse the DTI data to establish a statistically significant correlation between a relative change in symptom scores measured before, during and/or after treatment and a relative change in the DTI data obtained before, during and/or after treatment is selected from multivariate logistic regression, univariate logistic regression and linear discrimant analysis. However, the skilled reader will understand that the analysis of the DTI data to establish the statistically significant correlation can be performed using other routine and well-known statistical methods.

### Prediction of Remission

Test Development

This test was developed as part of the above-described clinical study to identify pre-treatment measures that predict or moderate MDD treatment outcomes to three commonly prescribed antidepressant drugs: escitalopram, sertraline or venlafaxine-XR. The imaging study is the first to test predictors such as DTI of treatment outcomes for one or several ADMs (escitalopram, sertraline, venlafaxine-XR) within a practical trial design. As indicated above, the study is a prospective trial assessing predictors of treatment outcome with a practical trial design in which MDD patients were assessed before and 8 weeks after randomization to escitalopram, sertraline or venlafaxine-XR. Imaging with DTI was undertaken pre-treatment. Patients were recruited from an academic psychiatry setting, with recruitment from community and primary care physicians. 102 MDD patients underwent imaging, with 34 matched patients in each of the 3 treatment arms.

Image Acquisition

Magnetic Resonance Images were acquired using a 3.0 Tesla GE Signa HDx scanner (GE Healthcare, Milwaukee, Wis.). Acquisition was performed using an 8-channel head coil. Diffusion tensor images were acquired using a spin-echo DTI-Echo Planar Imaging sequence, Seventy contiguous 2.5 mm slices were acquired in an axial orientation with an in-plane resolution of 1.72 mm×1.72 mm and a 128×128 matrix (TR: 17000 ms; TE: 95 ms; Fat Saturation: ON; NEX: 1; Frequency direction: R/L). A baseline image (b=0) and 42 different diffusion orientations were acquired with a b-value of 1250.

Tract-Based spatial Statistical Analysis of DTI Data

Details of the DTI data processing and analysis have been described in detail in Korgaonkar at al 2012 (Korgaonkar M S, Cooper N J, Williams L M, Grieve S M: Mapping inter-regional connectivity of the entire cortex to characterize major depressive disorder: a whole-brain diffusion tensor imaging tractography study. Neuroreport 2012:566-571 and Korgaonkar M S, Grieve S M, Koslow S H, Gabrieli J D E, Gordon E, Williams L M: Loss of white matter integrity in major depressive disorder: evidence using tract-based spatial statistical analysis of diffusion tensor imaging. Human brain mapping 2011, 32:2161-2171). Specifically, the DTI data was pre-processed and analysed using the Oxford Centre for Functional MRI of the Brain (FMRIB) Diffusion Toolbox (FDT) and TBSS software tools as part of the FMRIB Software Library (FSL) release 4.1.3 (http://www.fmrib.ox.ac.uk/fsl; Behrens T E J, Johansen-Berg H, Woolrich M W, Smith S M, Wheeler-Kingshott CaM, Boulby Pa, Barker G J, Sillery E L, Sheehan K, Ciccarelli O et al: Non-invasive mapping of connections between human thalamus and cortex using diffusion imaging. Nature neuroscience 2003, 6:750-757.; Smith S M: Fast robust automated brain extraction. Human brain mapping 2002, 17:143-155; Smith S M, Jenkinson M, Johansen-Berg H, Rueckert D, Nichols T E, Mackay C E, Watkins K E, Ciccarelli O, Cadet M Z, Matthews P M at a Tract-based spatial statistics: voxelwise analysis of multi-subject diffusion data. PleuraImage 2006, 31:1487-1505; Smith S M, Jenkinson M, Woolrich M W, Beckmann C F, Behrens T E J, Johansen-Berg H, Bannister P R, De Luca M, Drobnjak I, Flitney D E et al: Advances in functional and structural MR image analysis and implementation as FSL. NeuroImage 2004, 23 Suppl 1:S208-219).

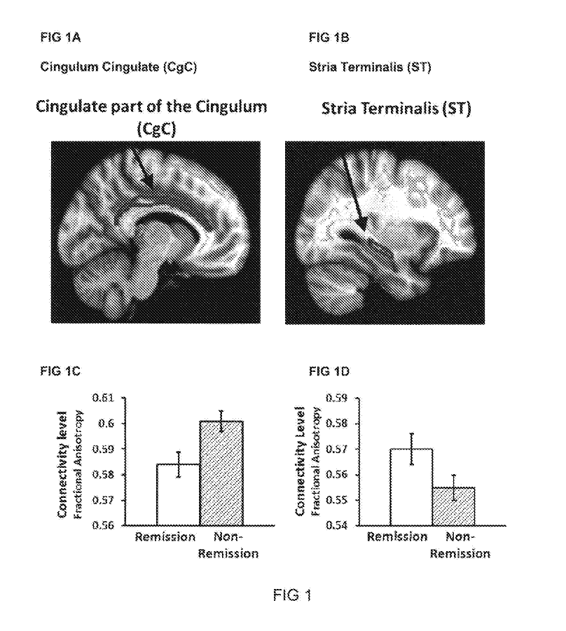

Diffusion tensor models were fitted and images of fractional anisotropy (FA) were generated for each participant. An average FA image was generated and thinned to create a WM skeleton representing the centres of all WM tracts common to all participants. This FA skeleton was then thresholded to FA ≧0.3 to include the major WM pathways but to avoid peripheral tracts. The Johns Hopkins University International Consortium for Brain Mapping (JHU ICBM)-DTI-81 white matter labels atlas was used to identify parts of the tract skeleton for the hypothesized fronto-limbic WM tracts (see FIG. 1) (Mori S, Oishi K, Jiang H, Jiang L, Li X, Akhter K, Hua K, Feria A V, Mahmood A, Woods R et al: Stereotaxic white matter atlas based on diffusion tensor imaging in an ICBM template. NeuroImage 2008, 40(2):570-582). Mean FA for each tract was calculated and used for further analyses.

Statistical Analyses: Prediction Models for Remission and Response Outcomes

Logistic regression statistics were used to test the prognostic utility of DTI FA measures to ADM treatment. Age, baseline depression severity, age of onset and duration of illness were found to be associated with treatment outcome (see Table 1). To remove any proxy effects of these measures via white matter FA, residual FA values were calculated controlling for these measures and used for all further analyses. MDD participants were characterized with regard to: (1) remission to treatment and (2) response to treatment and based on the criteria outlined above. These outcomes were tested in separate analyses as dependent variables.

Clinical outcome was remission defined by HDRS-17, administered by a clinician at the baseline and 8-week follow up session. Predictors were the fractional anisotropy (FA) index of WM integrity for five a priori tracts relevant to the anterior cingulate and limbic circuitry: the cingulate portion of the cingulum (CgC) and hippocampus (CgH), the stria terminalis (ST), the fornix (FX) and the uncinate fasciculus (UNC).

First step logistic regressions were performed for each of the hypothesized WM tracts (CgC, CgH, ST, FX, UNC) as independent variables to test prognostic value for each individual tract. As next step, all five WM tracts were pooled and tested in a single model using a backward stepwise logistic regression model. This analysis performs a backward stepwise elimination of candidate independent variables until no further improvement in model estimation. Both models were assessed using a significance level of p<0.05 and accuracy in prediction reported. A backward stepwise logistic regression model using all JHU ICBM-DTI-81 WM atlas labelled tracts was also analysed to confirm validity of findings for the pre-selected WM tracts when tested at the whole brain level.

These predictive models were re-run for patients stratified by the three separate treatment arms.

Cross-Validation of Prediction Models

To test generalization of models, a stepwise linear discriminant analysis (LDA) with an inbuilt K-fold cross-validation method was performed using the klaR package. In this cross-validation method, a random subset according to ratio K:1 is taken out and used as the testing set. The remaining dataset serves as training subset. The prediction models were derived using the training dataset and evaluated on the test dataset. The cross validated accuracy is the average of the accuracies of all randomizations. The cross-validation analysis was run for five different values of K: 2,3,5,10,15, which correspond to training sets of size: 50%, 66.6%, 80%, 90% and 95% respectively. Each analysis was performed 20 times and model accuracies (and the selected predictors) were combined across all 100 runs of stepwise LDA.

As next step, the five WM tracts were ranked based on prediction capacity. For each of the WM predictors, a summary score of model accuracy for models (out of all 100 runs) of which that predictor was a member was derived. The score for each trial is the total accuracy of the model containing that predictor. A predictor gets a higher score if it is part of more accurate models, and if it is more often present in the models (the maximum possible score for a predictor is 1). This score gives a measure of how often a particular predictor appeared in all final stepwise models and how accurate these models were. The accuracy percentages thus reflect the cross validated accuracies. The range of model accuracies provides an interval of expected cross-validated performance.

Next, models were constructed using the complete dataset, starting with a model containing only the best predictor (based on the scores from previous step), then adding the next best and so on until all predictors were included. Performance statistics were calculated for each of these ranked models.

Sensitivity, specificity, positive predictive power (PPP), negative predictive power (NPP), total accuracy, bayes error rate and correctness rate (1-error rate) were used to evaluate model performance. The bayes error rate uses the distribution of the linear composites of each model to determine how likely replication would be in an unseen test set. The bayes error shows a lower bound for the error rate or the best likely performance for the model. For the prediction model to hold replication in an unseen test dataset the correctness rate and the overall accuracy should be similar and the bayes error rate should be lower than that for chance i.e. 50% chance of categorizing into either group.

Patient Characteristics

The clinical and demographic features and remission and response rates for MDD patients are presented in Table 1. Of the 102 MDD participants, 59.8% were defined as responders and 39.2% as remitters based on our predefined criteria. Treatment responders were found to be younger, had a higher depression severity at baseline visit and had developed depression at a younger age in comparison to non-responders. Similarly, remitters were characterized by younger age and had suffered depression for a shorter period of time (see Table 1). As our primary interest in this study was to evaluate the role of WM tracts as predictors of outcome, these factors were regressed out to allow this be assessed independent of these factors (see Methods for details).

Predictors of Treatment Remission

FA for the CgC and ST bundle were found to be significant predictors of remission status with MDD participants showing higher FA in CgC and lower FA in ST proving to be more likely to achieve remission (p<0.05; see Table 2). In the univariate analysis, accuracy in prediction was better for CgC (overall 71.6% with 91.4% specificity and 40.5% sensitivity) in comparison to ST (overall 58.9% with 84.5% specificity and 18.9% sensitivity). Using all five pre-selected tracts together, the backward logistic regression analysis converged on a model containing only these two WM tracts (p=0.001). This model was 65.3% accurate overall, with a specificity of 79.3% and a sensitivity of 43.2% in remission prediction. Using a model starting with all major WM tracts from the JHU ICBM-DTI-81 WM atlas, a backwards stepwise regression identified the same predictive model with only the CgC and ST tracts selected (in the most parsimonious model).

The LDA cross-validation analysis validated the findings from the logistic regression analysis. The weighted prediction scores for each pre-defined white matter tract were: CgC=0.684; ST=0.496; FX=0.588; CgH=0.173; and UNC=0.179. CgC, ST and FX scored the highest, while the CgH and UNC were less important for prediction of remission status.

The prediction models using the three highest identified WM tracts (I.e. CgC, ST and FX) had the best classification statistics (Sensitivity=73%; Specificity=69%; overall accuracy=70.5%; PPP=0.6; NPR=0.8; correctness rate=67.7%) with the lowest bayes error rate (32.3%). The cross-validated correctness rate for this model was 68.5% (range=64.1-71.6%). The similar overall accuracy and correctness rate and the low bayes error rate indicate that this model is reliable for an independent cohort.

Predictors of Treatment Response

Out of the five pre-selected WM tracts, only FA for the FX was found to be a significant predictor of treatment response with MDD participants showing lower FA in FX proving to be more likely to respond to treatment (p=0.031; see Table 2). Using all five pre-selected tracts together, the backward logistic regression analysis converged on a model with only FX and CgC VVM tracts are listed In Table 2. A whole brain backward logistic analysis (as described above for remission) validated this model with these two tracts comprising the most parsimonious model—a result that further validates the role of both the FX and CgC tracts in the prediction of treatment response.

The LDA cross-validation analysis also confirmed FX as the primary predictor of response. The weighted scores for all five preselected tracts were: CgC=0.0798; ST=0.0673; FX=0.5975; CgH=0.0544; and UNC=0.0297. The cross-validation classification statistics for the prediction models using FX were: Sensitivity=54.4%; Specificity=73.7%; overall accuracy=62,1%; PPP=0.756; NPP=0.519; correctness rate=50.7%; bayes error rate=49.3%. The cross-validated correctness rate for this model was 61% (range=57.9%-62.8%). Although the overall accuracy and cross-validation rate were very similar, the bayes error rate for the model was close to chance (50% chance of falling in responder or non-responder group) suggesting poor reliability of this model in predicting treatment response in an independent cohort.

TABLE 2
Prediction of Response/Remission for the whole MDD group

| | | Overall model | | |
|---|---|---|---|---|
| | | summary | | Prediction accuracy |
| | | (Nagelkerke | Model Parameters | (% sensitivity/ |
| Models | | R² & p value) | (betas; p value) | % specificity/% overall) |
| RESPONSE | | | | |
| Individual pre- | FX | R² = 0.077; | FX: −7.47 (p = 0.031) | 80.7%/21.1%/56.8% |
| selected WM | | p = 0.019 | Constant: 0.45 | |
| tracts* | | | (p = 0.041) | |
| Pooled pre- | | R² = 0.114; | FX: −9.66 (p = 0.012) | 78.9%/28.9%/58.9% |
| selected WM | | p = 0.015 | CgC: 13.18 (p = 0.098) | |
| tracts (FX, | | | Constant: 0.46 | |
| CgC, CgH, ST, | | | (p = 0.038) | |
| UNC) | | | | |
| REMISSION | | | | |
| Individual pre- | CgC | R² = 0.077; | CgC: 17.11 (p = 0.024) | 40.5%/91.4%/71.6% |
| selected WM | | p = 0.018 | Constant: −0.48 | |
| tracts\hat{ } | | | (p = 0.028) | |
| | ST | R² = 0.054; | ST: −12.40 (p = 0.056) | 18.9%/84.5%/58.9% |
| | | p = 0.050 | Constant: −0.47 | |
| | | | (p = 0.030) | |
| Pooled pre- | | R² = 0.195; | CgC: 26.48 (p = 0.002) | 43.2%/79.3%/65.3% |
| selected WM | | p = 0.001 | ST: −21.99 (p = 0.005) | |
| tracts (FX, | | | Constant: −0.53 | |
| CgC, CgH, ST, | | | (p = 0.023) | |
| UNC) | | | | |

*FA for UNC/ST/CgC/CgH were not significant predictors of response;
\hat{ }FA for FX/UNC/CgH were not significant predictors of remission

Analysis of treatment predictuion by treatment arm:

Remission

When the five pre-selected WM tracts were analysed by individual treatment type, significant prediction of remission was seen only for MDD patients prescribed with venlafaxine-XR. For this treatment remission was predicted by the FA of the CgC and ST tracts only (p<0,05). As for the entire MDD cohort - higher FA in the CgC and a lower FA in the ST was associated with positive treatment outcome. Overall accuracy for remission was 75.8% (specificity=85%; sensitivity=61.5%).

The LDA analysis confirmed the CgC and ST as strong predictors of remission status for all three treatment groups (although not significant for escitalopram and sertraline group) with the highest predictor scores for these tracts for venlafaxine-XR. The classification statistics for the model using CgC and ST for the venlafaxine-XR group were: Sensitivity=76.9%; Specificityz=759%; overall accuracy=75.8%; PPP=0.667; NPP=0.833; correctness rate=66.2%; bayes error rate=33.8%.

Prediction scores for all five WM tracts for each treatment arm are as follows:

Escitalopram: CgC=0.404; ST=0.269; FX=0.224; CgH=0.082; UNC=0.377

Sertrafine: CgC=0.646; ST=0.336; FX=0.159: CgH=0.154; UNC=0.428

Venlafaxine-XR: CgC=0.625; ST=0.704; FX=0.182; CgH=0.434; UNC=0.144

Response

Significant results were found for response to only venlafaxine-XR, again with FA of the CgC (higher FA) and ST tracts (lower FA) the only significant predictors (p<0.05). The prediction of response with this model showed lower overall accuracy compared to remission with an overall accuracy of 63.6% (specificity=50%; sensitivity=73.7%).

The LDA analysis confirmed the CgC and ST as strong predictors of response status for venlafaxine-XR group. However the prediction scores were not on the same level as those for remission. The classification statistics using this model for the venlafaxine-XR group were: Sensitivity=63.2%; Specificity=71.4%; overall accuracy=66.7%; PPP=0.75; NPP=0.588; correctness rate=65.1%; bayes error rate =34.9%.

Prediction scores for all five WM tracts for each treatment arm are as follows:

Escitelopram; CgC=0.1174; ST=0.1007; FX=0.0606; CgH=0.0756; UNC=0.5038

Sertraline; CgC=0.1095; ST=0.2089; FX=0.6308; CgH=0.0453; UNC=0.0752

Venlafaxine-XR: CgC=0.535; ST=0.293: FX=0.280: CgH=0.166; UNC=0.094

TABLE 3
Prediction of Response/Remission for individual treatment types

| | | | | Prediction accuracy |
|---|---|---|---|---|
| | | | | (% sensitivity/ |
| | | Model R² & p | | % specificity/ |
| Models | Treatment | value | Significant Parameters | % overall) |
| RESPONSE | Escitalopram | NS | NS | — |
| Pooled pre- | Sertraline | NS | NS | — |
| selected WM | Venlafaxine- | R² = 0.274: | ST: −29.3 (p = 0.036) | 73.7%/50%/63.6% |
| tracts (FX, CgC, | XR | p = 0.023 | CgC: 35.7 (p = 0.045) | |
| CgH, ST, UNC) | | | Constant: 0.63 (p = 0.155) | |
| REMISSION | Escitalopram | NS | NS | — |
| Pooled pre- | Sertraline | NS | NS | — |
| selected WM | Venlafaxine- | R² = 0.270; | ST: −29.3 (p = 0.037) | 61.5%/85%/75.8% |
| tracts (FX, CgC, | XR | p = 0.025 | CgC: 33.8 (p = 0.053) | |
| CgH, ST, UNC) | | | Constant: −0.31 (p = 0.457) | |

Cingulate and limbic WM connectivity measured using DTI is a useful predictor of whether individual MDD patients are likely to respond to venlafaxine-XR. This imaging biomarker has potential for clinical translation and guiding individualized treatment choices for MDD.

### Prediction of Non-Remission

Image Acquisition

Magnetic Resonance Images were acquired using a 3.0 Tesla GE Signa HDx scanner (GE Healthcare, Milwaukee, Wis.), Acquisition was performed using an 8-channel head coil. Diffusion tensor images were acquired using a spin-echo DTI-Echo Planar Imaging sequence. Seventy contiguous 2.5 mm slices were acquired in an axial orientation with an in-plane resolution of 1.72 mm×1.72 mm and a 128×128 matrix (TR: 17000 ms; TE: 95 ms; Fat Saturation: ON; NEX: 1; Frequency direction: R/L). A baseline image (b=0) and 42 different diffusion orientations were acquired with a b-value of 1250.

Tract-Based Spatial Statistical Analysis of DTI Data

Details of the DTI data processing and analysis have been described in detail in Korgaonkar et al 2012 (Korgaonkar M S, Cooper N J, Williams L M, Grieve S M: Mapping inter-regional connectivity of the entire cortex to characterize major depressive disorder: a whole-brain diffusion tensor imaging tractography study. Neuroreport 2012:566-571 and Korgaonkar M S, Grieve S M, Koslow S H, Gabrieli J D E, Gordon E, Williams L M; Loss of white matter integrity in major depressive disorder: evidence using tract-based spatial statistical analysis of diffusion tensor imaging. Human brain mapping 2011, 32:2161-2171). Specifically, the DTI data was pre-processed and analysed using the Oxford Centre for Functional MRI of the Brain (FMRIB) Diffusion Toolbox (FDT) and TBSS software tools as part of the FMRIB Software Library (FSL) release 4.1.3 (http://www.fmrib.ox.ac.uk/fsl; Behrens T E J, Johansen-Berg H, Woolrich M W, Smith S M, Wheeler-Kingshott CaM, Bouiby Pa, Barker G J, Siliery E L, Sheehan K, Ciccarelli O et al: Non-Invasive mapping of connections between human thalamus and cortex using diffusion imaging. Nature neuroscience 2003, 6:750-757.; Smith S M: Fast robust automated brain extraction. Human brain mapping 2002, 17:143-155; Smith S M, Jenkinson M, Johansen-Berg H, Rueckert D, Nichols T E, Mackay C E, Watkins K E, Ciccarelli O, Ceder M Z, Matthews P M et al: Tract-based spatial statistics: voxelwise analysis of multi-subject diffusion data. NeuroImage 2006. 31:1487-1505; Smith S M, Jenkinson M, Woolrich M W, Beckmann C F, Behrens T E J, Johansen-Berg H, Bannister P R, De Luca M, Drobnjak I, Flitney D E et al: Advances in functional and structural MR image analysis and implementation as FSL. NeuroImage 2004, 23 Suppl 1:S208-219).

Diffusion tensor models were fitted and images of fractional anisotropy (FA) were generated for each participant. An average FA image was generated and thinned to create a WM skeleton representing the centres of all WM tracts common to all participants. This FA skeleton was then thresholded to FA ≧0.3 to include the major WM pathways but to avoid peripheral tracts. The Johns Hopkins University International Consortium for Brain Mapping (JHU ICBM)-DTI-81 white matter labels atlas was used to identify parts of the tract skeleton for the hypothesized fronto-limbic WM tracts (see FIG. 1) (Mori S, Oishi K, Jiang H, Jiang L, Li X, Akhter K, Hua K, Feria A V, Mahmood A, Woods R et al: Stereotaxic white matter atlas based on diffusion tensor imaging in an ICBM template. NeuroImage 2008, 40(2):570-582). Mean FA for each tract was calculated and used for further analyses.

Statistical Analyses

A combined measure of the stria terminalis and cingulate portion of cingulate bundle was created by calculating the ratio of these values (R_{ST-Cing}) for each patient (stria terminalis numerator, cingulate bundle denominator). A threshold value of R_{ST-Cing}=1.0 was then applied to the data, the specificity of this measure for the identification of non-responders was then calculated.

Prediction of Non-Remission

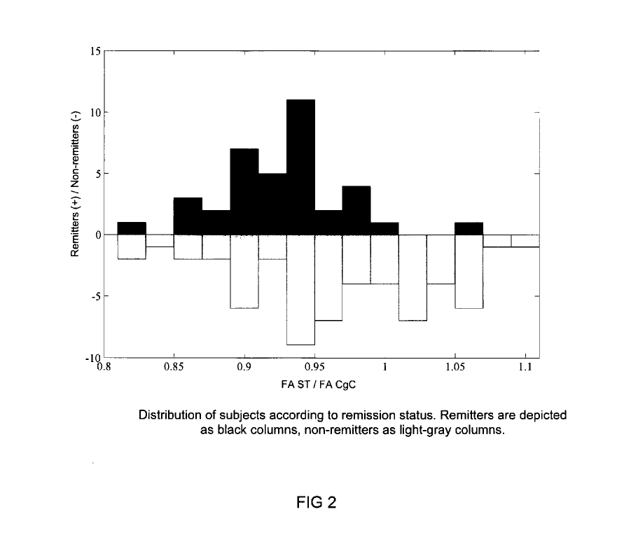

FIG. 2 shows the distribution of R_{ST-Cing} for both the remitter and non-remitting patients. Both groups do not significantly deviate from a normal distribution (stat here). The remitting population is skewed to the left (0.254), while the non-remitting population has a distribution slightly skewed to the right (−0.256). R_{ST-Cing} was significantly lower for remitters compared to non-remitters (0.93±0.05 versus 0.97±0.07, p<0.002). Using a threshold of R_{ST-Cing}>1.0 to select the non-remitter group, 31% of the overall non-remitters and 3% of overall remitters were selected. This corresponds to an NPV of 91%.

Characteristics of Selected Non-Remitter Group Versus Non-Selected Non-Remitters

The clinical and demographic characteristics of the selected non-remitter (S-NR) group were compared to the non-selected NR (N-NR) group. The only significant difference between these groups was age at first visit (S-NR: 32.1 vs. 40.1, p=0.029). No difference in age of diagnosis, disease duration, gender, baseline severity or percentage change in HRSD17 at week 8 was present. Secondary analyses were also performed comparing the S-NR groups with the remitter group and also with the entire non-selected group. The only significant differences observed were for severity (S-NR vs. remitters, p<0.001; S-NR vs. rest of group, p=0.027), for percentage HRSD17 change at week 8 (S-NR vs. remitters, p<0.001; N-NR vs. rest of group, p=0.034), and for side effects (FIBSER intensity) in between S-NR and remitters (p=0.006).

TABLE 2
Characteristics of selected non-remitting subjects
compared to non-selected subject groups

| | Selected | Rest of | | Non-selected | | | |
|---|---|---|---|---|---|---|---|
| | NRs | group | | NRs | | Remitters | |
| N | 21 | 74 | | 37 | | 35 | |
| Age | 32.1 | 34.8 | NS | 40.8 | 0.029 | 29 | NS |
| Females (%) | 61.9% | 43.2% | NS | 43.2% | NS | 42.9% | NS |
| HRSD17Baseline | 20.7 | 20.6 | NS | 20.4 | NS | 20.8 | NS |
| HRSD17 Week 8 | 11.1 | 8.6 | 0.027 | 12.6 | NS | 4.6 | p < 0.001 |
| HRSD17 % change | 44.4 | 57.1 | 0.034 | 37.2 | NS | 77.2 | p < 0.001 |
| Age of Onset | 21.8 | 22.1 | NS | 25.1 | NS | 19.2 | NS |
| Disease duration | 9.7 | 12.2 | NS | 15.4 | NS | 9.3 | NS |
| HDRS17 anxiety | 6.9 | 6.6 | NS | 6.6 | NS | 6.6 | NS |
| HDRS17 non-anxiety | 13.8 | 14 | NS | 13.7 | NS | 14.1 | NS |
| FIBSER (intensity) | 1.19 | 0.99 | NS | 1.4 | NS | 0.57 | 0.006 |
| MINI-Plus | 19.0% | 32.4% | NS | 37.8% | NS | 25.7% | NS |
| Melancholic | | | | | | | |

“HDRS17 anxiety” and “HDRS17 non-anxiety” refer to the degree of anxiety measured by the HDRS17 scale and referred to as the defining component of the “anxiety” and “non-anxiety” sub-types of MDD patients, respectively.
“FIBSER intensity” refers intensity component of side effects evaluated in the Frequency, Intensity and Burden of Side Effects Ratings questionnaire (Wisniewski S R, et al. J Psychiatr Pract. 2006; 12: 71-79.)
“MINI-Plus melancholic” refers to the MDD melancholia component as measured by the MINI-Plus scale and referred to as the defining component of the “melancholic” sub-type of MDD patients.

Discussion

In this report, a DTI-derived measure of connectivity to identify individual, depressed patients who did not remit after up to 8 weeks of ADM administration was used. This biomarker was derived from the ratio of the FA of the stria terminalis and the cingulate portion of the cingulate bundle. The stria terminalis and the cingulate portion of the cingulate bundle have previously been investigated in the context of MDD. Using this ratio, excellent separation of a large proportion of non-remitters, with 30% of non-remitting subjects able to be identified (19% of subjects overall in our study), corresponding to an NPV of 91%, can be shown, As indicated above, treatment of patients with ADMs is a trial-and-error process, and it may take many months to identify ultimately that the depression is resistant to standard pharmacotherapy. The identification of individuals who are very unlikely to remit with such treatment is therefore clinically valuable, as it serves to avoid unnecessary and expensive treatment for a sizeable fraction of the MDD population.

The non-remitting subjects that were selected using R_{ST-Cing} showed no differences in clinical characteristics compared to the non-selected non-remitters. These characteristics included disease duration, severity, change in severity, side-effect severity, or indices of anxiety and melancholia. No gender differences were present; however, there was a significant age difference, with the non-selected group tending to be older. Previously, the inventors used DTI to investigate white matter integrity data in the context of MDD (Korgaonkar M S, Cooper N J, Williams L M, Grieve S M: Mapping inter-regional connectivity of the entire cortex to characterize major depressive disorder: a whole-brain diffusion tensor imaging tractography study. Neuroreport 2012:566-571; Korgaonkar M S, Grieve S M, Koslow S H, Gabrieli J D E, Gordon E, Williams L M: Loss of white matter integrity in major depressive disorder: evidence using tract-based spatial statistical analysis of diffusion tensor imaging. Human brain mapping 2011, 32:2161-2171). From the present study, it is now clear that, surprisingly, the ratio of FA values for the stria terminalis and FA values for the cingulate portion of the cingulum of below 1 reflects a connectivity pattern that is very strongly associated with non-remission in a sub-set of individuals. This association appears independent of many indices that are normally used to sub-type depressive patients.

The inventors' data highlight the inability of standard clinical measures for understanding the heterogeneity present within the diagnosis of depression. Specifically, the baseline analyses of DTI data show that compared to controls, MDD participants have significant alterations in the CgC and the fornix but not the ST. Functional MRI data comparing controls and MDD subjects also highlight abnormal activation patterns in the amygdala (Korgaonkar M S, Grieve S M, Etkin A, Koslow S H, Williams L M: Using Standardized fMRI Protocols to identify Patterns of Prefrontal Circuit Dysregulation that are Common and specific to Cognitive and Emotional Tasks in Major Depressive Disorder: First wave Results from the iSPOT-D study. Neuropsychopharmacology 2012) and sub-genual ACC (Mayberg H S, Brannan S K, Mahurin R K, Jerabek Pa, Brickman J S, Tekell J L, Silva Ja, McGinnis S, Glass T G, Martin C C et al: Cingulate function in depression: a potential predictor of treatment response. Neuroreport 1997, 8:1057-1061; Whalen P J, Johnstone T, Somerville L H, Nitschke J B, Polls S, Alexander A L, Davidson R J, Kalin N H: A functional magnetic resonance imaging predictor of treatment response to venlafaxine-XR in generalized anxiety disorder. Biol Psychiatry 2008, 63(9):858-863; Siegle G J, Thompson W K, Collier A, Berman S R, Feldmihler J, Thase M E, Friedman E S: Toward clinically useful neuroimaging in depression treatment: prognostic utility of subgenual cingulate activity for determining depression outcome in cognitive therapy across studies, scanners, and patient characteristics. Arch Gen Psychiatry 2012, 69(9):913-924).

As indicated above, in some embodiments of the present invention, these two tracts can also be used in the prediction of remission. These results highlight that while both the CgC and ST (as the outflow tract of the amygdala) are abnormal in depression, the patterns of these differences and how they interact with treatment with ADMs is quite dissimilar. The CgC collects projections from the rostral prefrontal/anterior cingulate cortices to the posterior cingulate, while the fornix and ST are comprised of axonal projections from the hippocampus and the amygdala, respectively, and connect to the hypothalamus and the rest of the limbic system (Wakana S, Jiang H, Ziji PCMV: Radiology Fiber Tract-based Atlas of. Radiology 2003:21-29). Accordingly, the inventors' results allow for the sub-typing subjects with MDD in ways that are more clinically useful than traditional clinical measures by analysing brain connectivity using, for example, DTI or resting state MRI to.

In conclusion, about 30% of depressed outpatients with a high risk for non-remission were identified with a high level of specificity using a biologically plausible parameter that reflects connectivity in two tracts (CgC and ST) central to the development or maintenance of a depressed state. The prediction of non-remission with high specificity is a viable avenue to personalizing or accurately targeting treatment.

As used herein, the term “exemplary” is used in the sense of providing examples, as opposed to indicating quality. That is, an “exemplary embodiment” is an embodiment provided as an example, as opposed to necessarily being an embodiment of exemplary quality.

It should be appreciated that in the above description of exemplary embodiments of the invention, various features of the invention are sometimes grouped together in a single embodiment, FIG., or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

Similarly, it is to be noticed that the term coupled, when used in the claims, should not be interpreted as being limited to direct connections only. The terms “coupled” and “connected,” along with their derivatives, may be used. It should be understood that these terms are not intended as synonyms for each other. Thus, the scope of the expression a device A coupled to a device B should not be limited to devices or systems wherein an output of device A is directly connected to an input of device B. It means that there exists a path between an output of A and an input of B which may be a path including other devices or means. “Coupled” may mean that two or more elements are either in direct physical or electrical contact or that two or more elements are not in direct contact with each other but yet still co-operate or interact with each other.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

1. A method of determining a medical treatment outcome in a brain-related condition of a subject, the method including the steps of:
a) determining the state of the white matter circuits of the subject's brain; and
b) utilising said state of the white matter circuits to predict the likely medical treatment outcome.

2. The method as claimed in claim 1 wherein step a) includes conducting diffusion tensor imaging (DTI) of the subject's brain.

3. The method as claimed in claim 1 wherein step b) includes determining the state of anterior cingulate or the limbic circuits as a biomarker of the likely medical treatment outcome.

4. The method as claimed in claim 2 wherein the brain-related condition is Major Depressive Disorder (MDD).

5. The method as claimed in claim 4 wherein the treatment is a treatment for depression.

6. The method as claimed in claim 5 wherein the treatment for depression comprises the administration of an antidepressant medication (ADM).

7. The method as claimed in claim 6 wherein the ADM comprises venlafaxine-XR.

8. The method as claimed in claim 5 wherein treatment for depression comprises the administration of an ADM selected from escitalopram, sertraline and venlafaxine-extended release (venlafaxine-XR).

9. The method as claimed in claim 1 wherein the degree of connectivity of the cingulate portion of the cingulum and the stria terminalis are determined in step a).

10. A method of predicting symptom remission or symptom response in a patient with Major Depressive Disorder (MDD) when treated with a selected ADM comprising the steps of:
a) performing diffusion tensor imaging (DTI) of the cingulate gyrus and the stria terminalis to measure fractional anisotropy (FA) of the white matter circuits in these areas of the patient's brain; and
b) utilising said measure of step a) to predict symptom remission or symptom response,
wherein a ratio of FA values for the cingulate portion of the cingulum to FA values for the stria terminals below 1 is predictive of symptom remission.

11. A method of predicting non-remission in a patient with Major Depressive Disorder (MDD) when treated with a selected ADM comprising the steps of:
a) performing Diffusion Tensor Imaging (DTI) of the cingulate gyrus and the stria terminalis to obtain a measure of fractional anisotropy (FA) in these areas of the patient's brain; and
b) utilising said measure of step a) to predict non-remission,
wherein a ratio of FA values for the stria terminalis to FA values for the cingulate portion of the cingulum below 1 is predictive of non-remission.

12. A method of treating Major Depressive Disorder (MDD) in a patient, wherein said MDD is associated with a particular state of the white matter circuits of the patient's brain, said method comprising the steps of:
a) performing diffusion tensor imaging (DTI) to obtain a measure of fractional anisotropy (FA) in an area of the patient's brain;
b) comparing said measure of step a) with a reference set of FA measures obtained from MDD patients to establish a correlation between said measure of with a corresponding FA measure of the reference set;
c) selecting an antidepressant medication (ADM) based on said correlation of step b), and wherein said corresponding FA measure of the reference set has been linked to beneficial treatment outcome in MDD patients having been treated with said ADM; and
d) administering said ADM selected in step c) to said patient to treat said MDD.

13. The method as claimed in claim 12 wherein DTI of the cingulate gyrus and the stria terminalis is performed in step a).

14. The method as claimed in claim 13 wherein the ADM is selected from the group consisting of selective serotonin reuptake inhibitors (SSRIs) and serotonin reuptake inhibitors (SNRIs).

15. The method as claimed in claim 14 wherein the ADM is selected from escitalopram, sertraline and venlafaxine-extended release (venlafaxine-XR).
